(19) United States
(12) Patent Application Publication
(10) Pub. No.: US 2002/0111481 A1
(43) Pub. Date: Aug. 15, 2002

(54) Processes for the preparation of alphaGal(1-4)betaGal(1-4)Glc-OR trisaccharides

(75) Inventors: Murray R. Ratcliffe, Cochrane (CA); Jonathan M. Gregson, Calgary (CA); Vivek P. Kamath, Calgary (CA); Robert E. Yeske, Calgary (CA)

Correspondence Address: BURNS DOANE SWECKER & MATHIS L L P, POST OFFICE BOX 1404, ALEXANDRIA, VA, 22313-1404, US

(73) Assignee: Synsorb Biotech, Inc., NW Calgary (CA)

(21) Appl. No.: 09/986,642

(22) Filed: Nov. 9, 2001

Related U.S. Application Data
(60) Provisional application No. 60/255,989, filed on Dec. 15, 2000.
(60) Provisional application No. 60/259,024, filed on Dec. 29, 2000.

(51) Int. Cl. C07H 15/04

(52) U.S. Cl. 536/123.1; 536/124; 536/120

(57) ABSTRACT

Disclosed are novel synthetic processes for the preparation of αGal(1→4) βGal(1→4)Glc-OR trisaccharides.

### CROSS REFERENCE TO RELATED APPLICATIONS

This application claims priority to U.S. Provisional Patent Application Serial No. 60/255,989, filed Dec. 15, 2000 and U.S. Provisional Patent Application Serial No. 60/259,024, filed Dec. 29, 2000, both of which are incorporated herein in their entirety.

### BACKGROUND OF THE INVENTION

1. Field of the Invention

This invention is directed to novel synthetic processes for the preparation of αGal(1→4) βGal(1→4)Glc-OR trisaccharides. Specifically, this invention is directed to a multi-step synthesis of this trisaccharide aglycon wherein the attachment of the aglycon is conducted after formation of the blocked βGal(1→4)Glc-OR disaccharide.

2. References

The following publications, patent and patent applications are cited in this application as superscript numbers:

¹ Armstrong, et al., U.S. Pat. No. 5,620,858 for “Diagnosis and Treatment of Bacterial Dysentery”, issued Apr. 15, 1997.

² Ratcliffe, et al., U.S. Pat. No. 5,079,353, for “Sialic Acid Glycosides, Antigens, Immunoadsorbents, and Methods for Their Preparation” issued Jan. 7, 1992.

³ Ekborg, et al., “Synthesis of Three Disaccharides for the Preparation of Immunogens Bearing Immunodeterminants Known to Occur on Glycoproteins”, Carbohydr. Res., 110:55-67 (1982).

⁴ Dahmen, et al., “2-Bromoethyl Glycosides:Applications in the Synthesis of Spacer-Arm Glycosides Carbohydr. Res., 118:292-301. (1983)

⁵ Rana, et al., “Synthesis of Phenyl 2-Acetamido-2-Deoxy-3-O-α-L-Fucopyranosyl-β-D-Glucopyranoside and Related Compounds”, Carbohydr. Res., 91:149-157 (1981).

⁶ Amvam-Zollo, et al., “Type XIV Polysaccharide: Synthesis of a Repeating Branched Tetrasaccharide with Dioxa-Type Spacer-Arms” Carbohydr. Res., 150:199-212 (1986).

⁷ Paulsen, et al., “Synthese Von Oligosaccharid-Determinanten Mit Amid-Spacer Vom Typ Des T-Antigens*”, Carbohydr. Res., 104:195-219 (1984).

⁸ Chernyak, et al., “New Type of Carbohydrate-Containing Synthetic Antigen: Synthesis of Carbohydrate-Containing Polyacrylamide Copolymers Having the Specificity of O:3 and O:4 Factors of Salmonella”, Carbohydr. Res., 128:269-282 (1984).

⁹ Fernadez-Santana, et al., “Glycosides of Monoallyl Diethylene Glycol. A new Type of Spacer Group for Synthetic Oligosaccharides”, J. Carbohydr. Chem., 8:531-537 (1989).

¹⁰ Lee, et al., “Synthesis of 3-(2-aminoethylthio)propyl Glycosides”, Carbohydr. Res., 37:193 et seq., (1974).

¹¹ Rafter, et al., U.S. Pat. No. 5,849,714, for “Treatment of Bacterial Dysentery, issued Dec. 15, 1998

¹² Liptak, et al., Carbohydr. Res., 51:19 (1976)

¹³ Nilsson, et al., Carbohydr. Res., 252:117 (1994)

All of the above publications and patents are herein incorporated by reference in their entirety to the same extent as if each individual publication, patent or patent application was specifically and individually indicated to be incorporated by reference in its entirety.

3. State of the Art

Trisaccharide glycosides, such as the αGal(1→4)βGal(1→4)Glc-OR trisaccharide, have been disclosed by Armstrong, et al.¹ as binding to Shiga Like Toxins (SLTs) expressed by pathogenic E. coli which can populate the intestinal tract of humans and cause diarrhea. In extreme cases, the pathology of this disease mediated by such toxins progresses to kidney involvement in the form of hemolytic uremic syndrome (HUS) which has a relatively high mortality rate. Accordingly, pharmaceutical compositions comprising such trisaccharides have been proposed for treatment of diarrhea mediated by SLTs as well as in the prevention of HUS.¹¹

Notwithstanding the beneficial properties of such trisaccharides, current synthetic processes for these compounds involve a multi-step process with overall low yields. This, in turn, has hampered the commercial development of these compounds.

Specifically, the complete chemical synthesis of oligosaccharide glycosides is a difficult task involving the generation of differentially protected or blocked hydroxyl groups on at least some of the hydroxyl groups of each of the saccharide units so as to provide a means to selectively remove one or more of the blocking groups thereby permitting the necessary reactions to be conducted on the unblocked hydroxyl group(s) as required to generate the desired compound.² Additionally, the numerous reaction procedures required in blocking and deblocking different hydroxyl groups necessitate a multi-step chemical synthetic procedure and the generation of crystalline intermediates during the synthetic procedure is certainly desirable in providing a facile means to purify the intermediates other than by chromatography or other equivalent means. In this regard, chromatography on intermediates and products achieved by large scale synthesis of trisaccharide glycosides is recognized as a time consuming process which can require the use of expensive equipment and is generally disadvantageous to an efficient large scale overall synthesis of the desired trisaccharide glycoside.

Contrarily, the use of glycosyltransferases to effect overall synthesis of the desired trisaccharide glycoside can be hindered by the lack of ready availability of the required glycosyltransferase, the difficulty in effecting large scale enzymatic reactions, the difficulty in coupling the desired saccharide to the nucleotide base required for coupling, etc.

### SUMMARY OF THE INVENTION

This invention is directed to novel processes for the overall chemical synthesis of αGal(1→4)βGal(1→4)Glc-OR which processes involve the derivation of a readily available lactose disaccharide derivative. Specifically and contrary to prior art processes, in one aspect, the processes of this invention defer attachment of the aglycon substituent (i.e., the R group) until after the lactose disaccharide structure has been fully protected. Surprisingly, by so deferring such an attachment, the overall yields of this trisaccharide are significantly improved.

In addition to the above, some of the intermediates generated in the overall synthetic scheme of this invention are readily crystalline which further facilitates the overall synthetic process by eliminating chromatography steps which correspondingly facilitates the synthesis and can enhance the overall yield.

Additionally, in the herein described processes, the synthesis of the trisaccharide glycoside is completed in such a fashion that the number of manipulations at the disaccharide and trisaccharide levels is kept to a minimum and yield is improved.

Accordingly, in one of its process aspects, this invention is directed to a process for preparing αGal(1→4)βGal(1→4)Glc-OR compounds, and pharmaceutically acceptable salts thereof, which process comprises:

(a) contacting β-R-lactoside represented by the formula:

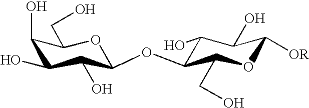

with at least a stoichiometric amount of a benzaldehyde dimethylacetal under conditions to provide for β-R-4′,6′-O-benzylidine lactoside of the formula:

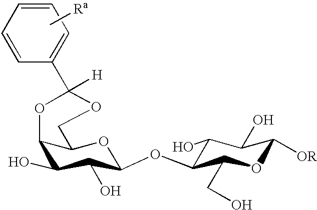

where R is an aglycon of at least 1 carbon atom and R^{a} is selected from the group consisting of hydrogen, alkyl, alkoxy, aryl, aryloxy, cyano and halo;

(b) acylating the compound produced in (a) above with at least 5 equivalents of an acyl halide under conditions to provide for β-R-4′,6′-O-benzylidine-2,2′,3,3′,6-pentaacyloxy-lactoside of the formula:

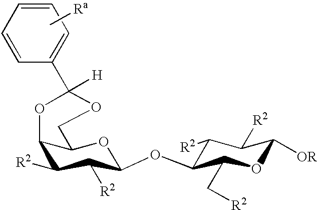

wherein R and R^{a} are as defined above and each R² is an acyloxy group;

(c) opening the benzylidine group from the compound produced in (b) above to provide for the β-R-2,2′,3,3′,6-pentaacyloxy-6′-O-benzyl lactoside of the formula:

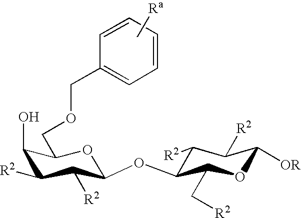

where R, R^{a} and R² are as defined above;

(d) contacting the compound produced in (c) above with α-chloro 2,3,4,6-tetra-O-benzyl-D-galactose under conditions to provide for a compound of the formula:

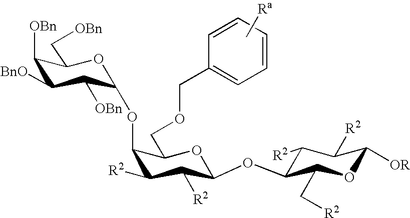

where R, R^{a} and R² are as defined above;

(e) removing each of the benzyl and R² protecting groups in the compound produced in (f) above under conditions to provide for αGal(1→4)βGal(1→4)Glc-OR which is represented by the formula:

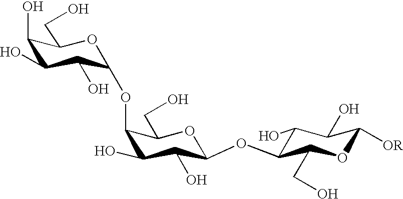

where R is as defined above.

Preferably, the β-R-lactoside represented by the formula:

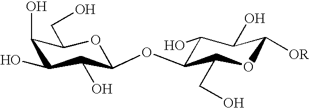

where R is as defined above, is prepared by the following process:

(i) contacting lactose of the formula:

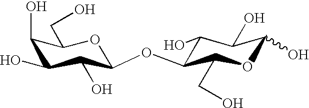

with at least 8 equivalents of benzoyl halide under conditions to provide for β-per-O-benzoyl-lactoside of the formula:

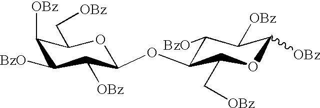

(ii) contacting the β-per-O-benzoyl-lactoside prepared in (i) above with at least a stoichiometric amount of hydrogen bromide under conditions to form the 1-α-bromo derivative of the formula:

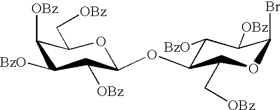

(iii) contacting the compound produced in (ii) above with a compound of the formula ROH under conditions to provide for β-R-2,2′,3,3′,4′,6,6′-hepta-O-benzoyl lactoside of the formula;

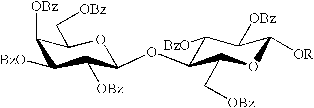

wherein R is an aglycon of at least 1 carbon atom; and

(iv) contacting the compound produced in (iii) above under conditions to debenzoylate said compound to provide for β-R-lactoside.

Preferably, the aglycon, R, contains from 1 to 20 carbon atoms and more preferably contains at least one functional group which allows attachment to a solid support. Most preferably, R is —(CH₂)₈COOCH₃.

Preferably, each R² is —O-benzoyl.

In another of its process aspects, this invention is directed to a process for the synthesis of αGal(1→4)βGal(1→4) Glc-O(CH₂)₈—COOCH₃ which process comprises:

(a) contacting lactose of the formula:

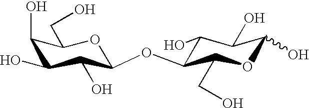

with at least 8 equivalents of benzoyl halide under conditions to provide for β-per-O-benzoyl-lactoside of the formula:

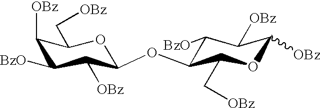

(b) contacting the β-per-O-benzoyl-lactoside prepared in (a) above with at least a stoichiometric amount of hydrogen bromide under conditions to form the 1-α-bromo derivative of the formula:

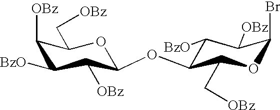

(c) contacting the compound produced in (b) above with at least a stoichiometeric amount of a compound of the formula ROH under conditions to provide for β-OR-2,2′,3,3′,4′,6,6′-hepta-O-benzoyl lactoside of the formula:

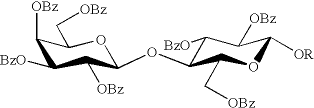

wherein R is —(CH₂)₈COOCH₃;

(d) contacting the compound produced in (c) above under conditions to debenzoylate said compound to provide for β-R-lactoside of the formula:

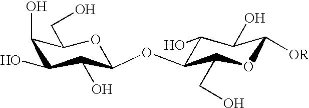

wherein R is as defined above;

(e) contacting β-R-lactoside produced in (d) with at least a stoichiometric amount of a benzaldehyde dimethylacetal under conditions to provide for β-R-4′,6′-O-benzylidine lactoside of the formula:

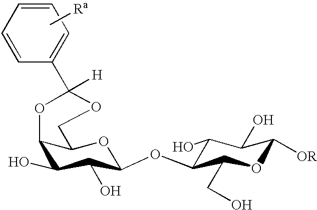

where R is an aglycon of at least 1 carbon atoms and R^{a} is selected from the group consisting of hydrogen, alkyl, alkoxy, aryl, aryloxy, cyano and halo;

(f) acylating the compound produced in (e) above with at least 5 equivalents of an acyl halide under conditions to provide for β-R-4′,6′-O-benzylidine-2,2′,3,3′,6,-pentaacyloxy-lactoside of the formula:

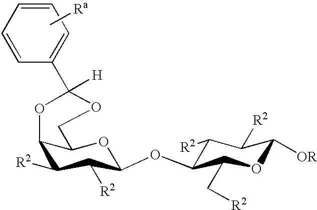

wherein R and R^{a} are as defined above and each R² is an acyloxy group;

(g) opening the benzylidine group from the compound produced in (f) above to provide for the β-R-2,2′,3,3′,6-pentaacyloxy-6′-O-benzyl lactoside of the formula:

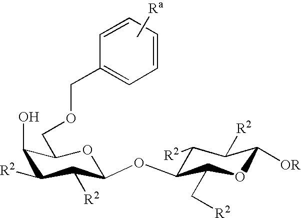

where R, R^{a} and R² are as defined above;

(h) contacting the compound produced in (g) above with α-chloro 2,3,4,6-tetra-O-benzyl-D-galactose under conditions to provide for a compound of the formula:

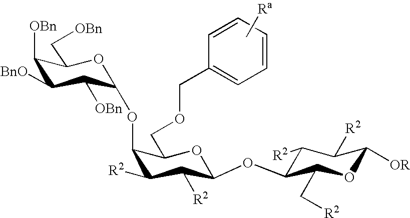

where R, R^{a} and R² are as defined above;

(i) removing each of the benzyl and R² protecting groups in the compound produced in (h) above under conditions to provide for αGal(1→4)βGal(1→4)Glc-OR which is represented by the formula:

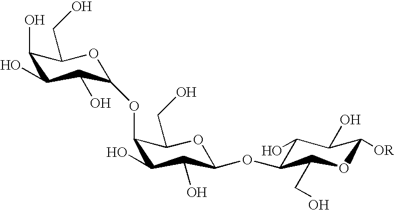

where R is as defined above.

### BRIEF DESCRIPTION OF THE DRAWINGS

FIG. 1 illustrates the preferred synthetic procedure for the preparation of 8-methoxycarbonyloctyl (α-D-galactopyranosyl)-(1→4)-O-(β-D-galactopyranosyl)-(1→4)-O-(β-D-glucopyranoside) starting with lactose. This compound is also referred to as αGal(1→4)βGal(1→4)Glc-OR or the Pₖ trisaccharide-OR where R is 8-methoxycarbonyloctyl.

### DETAILED DESCRIPTION OF THE INVENTION

This invention is directed to processes for the preparation of the trisaccharide αGal(1→4)βGal(1→4)Glc-OR.

Prior to discussing this invention in further detail, the following terms will first be defined:

The term “lactose” refers to the disaccharide βGal(1→4)Glc which can be represented by the formula:

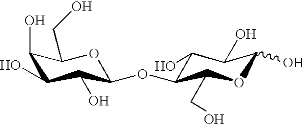

The term “lactoside” refers to the disaccharide βGal(1→4)Glc-OR where R is an aglycon of at least one carbon atom.

The term “aglycon of at least one carbon atom” refers to non-saccharide containing residues having at least one carbon atom, preferably from 1 to 20 carbon atoms and more preferably from 1 to 10 carbon atoms. Even more preferably, the aglycon is selected from the group consisting of —(A)—Z wherein A represents a bond, an alkylene group of from 2 to 10 carbon atoms, and a moiety of the form —(WG)ₙ— wherein n is an integer equal to 1 to 5; W is a straight or branched chain alkylene group of from 2 to 10 carbon atoms optionally substituted with 1 to 3 substituents selected from the group consisting of aryl of 6 to 10 carbon atoms and aryl of from 6 to 10 carbon atoms substituted with from 1 to 3 substituents selected from the group consisting of amino, hydroxyl, halo, alkyl of from 1 to 4 carbon atoms and alkoxy of from 1 to 4 carbon atoms; G is selected from the group consisting of a bond, O, S and NH; and Z is selected from the group consisting of hydrogen, methyl, phenyl, nitrophenyl and, when G is not oxygen, sulphur or nitrogen and when A is not a bond, then Z is also selected from the group consisting of —OH, —SH, —NH₂, —NHR³, —N(R³)₂, —C(O)OH, —C(O)OR³, —C(O)NH—NH₂, —C(O)NH₂, —C(O)NHR³, —C(O)N(R³)₂, and —OR⁴ wherein each R³ is independently alkyl of from 1 to 4 carbon atoms and R⁴ is an alkenyl group of from 3 to 10 carbon atoms.

Preferably, the aglycon contains a functional group or can be derivatized to contain a functional group which allows the aglycon to covalently bond to a solid support thereby providing for a compatible linker arm between the oligosaccharide and the solid support. Such functional groups are well known in the art and are selected to bind to a complementary functional group on the solid support to form a covalent bond or linkage. Such complementary functional groups include a first reactive group present on either the aglycon or the solid support and a second reactive group found on either the solid support or the aglycon and include, by way of example, those set forth below:

COMPLEMENTARY BINDING CHEMISTRIES

| First Reactive Group | Second Reactive Group | Linkage |
|---|---|---|
| hydroxyl | isocyanate | urethane |
| amine | epoxide | β-hydroxyamine |
| sulfonyl halide | amine | sulfonamide |
| carboxyl acid | amine | amide |
| hydroxyl | alkyl/aryl halide | ether |
| aldehyde | amine/NaCNBH₄ | amine |
| ketone | amine/NaCNBH₄ | amine |
| amine | isocyanate | urea |

Numerous aglycons are known in the art. For example, an aglycon comprising a p-nitrophenyl group (i.e., —OR═—OC₆H₄(p)NO₂) has been disclosed by Ekborg, et al.³ At the appropriate time during synthesis, the nitro group is reduced to an amino group which can be protected as N-trifluoroacetamido. When desired, the trifluoroacetamido group is removed thereby unmasking the amino group which can be used for coupling to a solid support.

An aglycon containing sulfur is disclosed by Dahmen, et al.⁴ Specifically, the aglycon is derived from a 2-bromoethyl group which, in a substitution reaction with thionucleophiles, has been shown to lead to aglycons possessing a variety of terminal functional groups such as —OCH₂CH₂SCH₂CO₂CH₃ and —OCH₂CH₂SC₆H₄—p-NH₂ both of which can be used to couple this aglycon to a solid support.

Rana, et al.⁵ discloses a 6-trifluoroacetamidohexyl aglycon [—O(CH₂)₆NHCOCF₃] in which the trifluoroacetamido protecting group can be removed unmasking the primary amino group which, again, can be used to couple this aglycon to a solid support.

Other exemplifications of known aglycons include the 7-methoxycarbonyl-3,6-dioxaheptyl aglycon⁶ (—OCH₂CH₂)₂OCH₂CO₂CH₃; the 2-(4-methoxycarbonylbutancarboxamido)ethyl⁷ [—OCH₂CH₂NHC(O)(CH₂)₄CO₂CH₃]; and the allyl aglycon⁸ (—OCH₂CH═CH₂) which, by radical co-polymerization with an appropriate monomer, leads to co-polymers. Other allyl linking aglycons⁹ are known [e.g., —O(CH₂CH₂O)₂CH₂CH═CH₂]. Additionally, allyl linking arms can be derivatized in the presence of 2-aminoethanethiol¹⁰ to provide for the aglycon of the formula —OCH₂CH₂CH₂SCH₂CH₂NH₂. As before, such aglycons permit covalent linkage to a solid support containing reactive groups complementary to the groups on the aglycon. That is to say that a complementary reactive group on the solid support is one which will selectively react with a reactive functionality on the aglycon to provide for covalent linkage therebetween.

Additionally, as shown by Ratcliffe, et al.², the aglycon R¹ group can be an additional saccharide-OR⁴ or an oligosaccharide-OR⁴ at the reducing sugar terminus (where R⁴ is an aglycon as defined above).

Preferably, the aglycon moiety is a —(CH₂)₈COOCH₃.

The term “compatible linker arm” refers to a moiety which serves to space the oligosaccharide structure from the solid support and which is bifunctional wherein one functional group is capable of binding to a reciprocal functional group of the support and the other functional group is capable of binding to a reciprocal functional group of the oligosaccharide structure. Compatible linker arms preferred in the present invention are non-peptidyl spacer arms.

In a preferred embodiment, the aglycon attached to the oligosaccharide comprises functionality or can be derivatized to contain functionality which permits attachment of the aglycon to the solid support. For example, allyl groups, nitro groups and carboxyl esters can be derivatized via conventional synthetic methods to permit covalent linkage to a compatible functional group on the surface of a solid support. Epoxides, amines, hydrazines, and similar groups on the aglycon can be reacted directly with a compatible functional group on the surface of a solid support to effect covalent linkage.

The term “solid support” refers to an inert, solid material to which the oligosaccharide sequences may be bound via a compatible linker arm. Where use is in vivo, the solid support will be biocompatible and preferably non-immunogenic.

For purpose of this application, all sugars are referenced using conventional three letter nomenclature. All sugars are assumed to be in the D-form unless otherwise noted, except for fructose, which is in the L-form. Further all sugars are in the pyranose form.

The term “pharmaceutically acceptable salts” include any and all pharmaceutically acceptable addition salts of αGal(1→3)βGal(1→4)Glc-OR compounds derived from a variety of organic and inorganic counter ions well known in the art and include, by way of example only, sodium, potassium, calcium, magnesium, ammonium, tetraalkylammonium, and the like.

The term “removable blocking group” refers to any group which when bound to one or more hydroxyl groups of either or both galactose units or the glucose unit, used to prepare the αGal(1→4)βGal(1→4)Glc-OR compounds described herein, prevents reactions from occurring at these hydroxyl groups and which protecting groups can be removed by conventional chemical and/or enzymatic procedures to reestablish the hydroxyl group without otherwise unintentionally altering the structure of the compound. The particular removable blocking group employed is not critical and preferred removable hydroxyl blocking groups include conventional substituents such as benzyl, benzoyl, acetyl, chloroacetyl, benzylidene, t-butylbiphenylsilyl and any other group that can be introduced onto a hydroxyl functionality and later selectively removed by conventional methods in mild conditions compatible with the nature of the product.

As used herein, “alkyl” refers to alkyl groups preferably having from 1 to 6 carbon atoms and more preferably 1 to 4 carbon atoms. This term is exemplified by groups such as methyl, ethyl, n-propyl, iso-propyl, n-butyl, tert-butyl, sec-butyl, n-pentyl, n-hexyl, 1,2-dimethylbutyl, and the like.

“Alkoxy” refers to the group —O-alkyl where alkyl is as defined herein.

“Alkenyl” refers to alkenyl groups preferably of from 1 to 6 carbon atoms and more preferably from 1 to 4 carbon atoms having from 1 to 2 sites of vinylic unsaturation (i.e., >C═C<). Such groups are exemplified by allyl (—CH₂CH═CH₂).

“Aryl” refers to an unsaturated aromatic carbocyclic group of from 6 to 10 carbon atoms having a single ring (e.g., phenyl) or multiple condensed rings (e.g., naphthyl).

“Alkaryl” refers to alkyl groups containing 1 to 2 aryl substituents thereon. Such groups preferably comprise from 7 to 18 carbon atoms and are represented by benzyl, —CH₂CH₂-φ, —CH(φ)₂ and the like.

“Aryloxy” refers to the group —O-aryl where aryl is as defined herein.

“Acyloxy” refers to the group R⁵C(O)O— where R⁵ is alkyl, aryl, alkaryl and the like. In the processes of this invention, the R⁵ substituent of the acyloxy group is preferably benzyl.

“Acyl halide” refers to the group R⁵C(O)X where R⁵ is as defined above.

“Halo” or “halide” refers to chloro or bromo.

Methodology

The αGal(1→4)βGal(1→4)Glc-OR compounds described herein may be prepared by the following general methods and procedures. It should be appreciated that where typical or preferred process conditions (e.g., reaction temperatures, times, mole ratios of reactants, solvents, pressures, etc.) are given, other process conditions may also be used unless otherwise stated. Optimum reaction conditions may vary with the particular reactants or solvents used, but such conditions can be determined by one skilled in the art by routine optimization procedures.

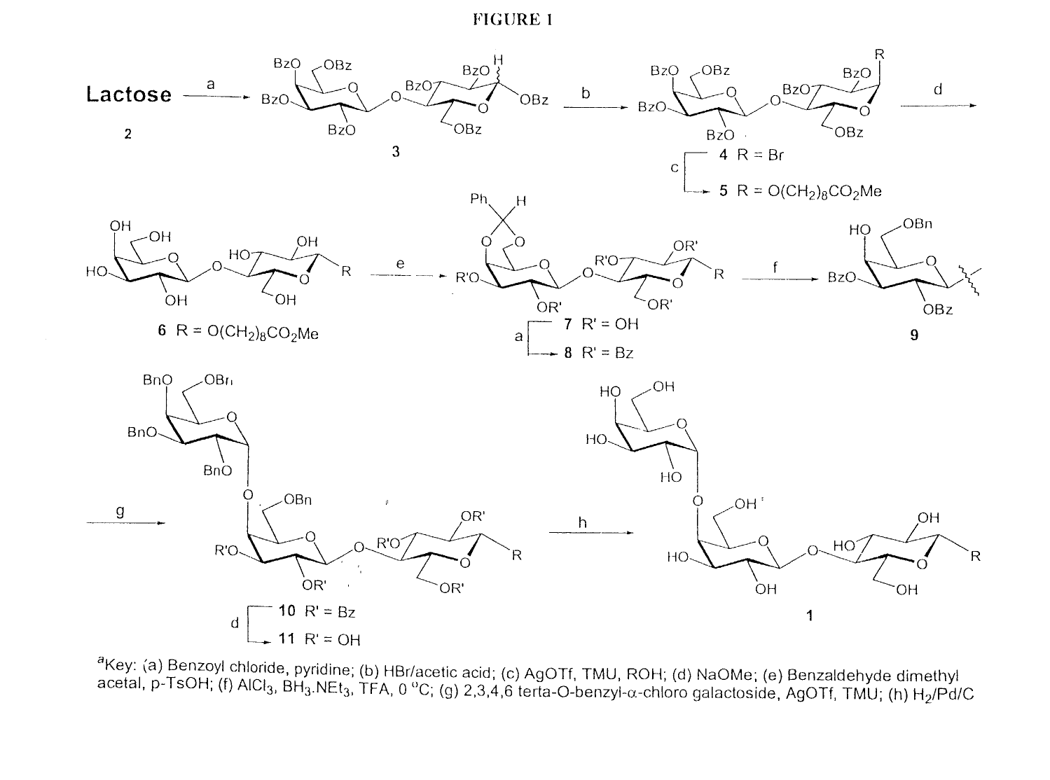

FIG. 1 illustrates preferred reaction schemes using a —(CH₂)₈COOCH₃ aglycon which is described in detail below. It is understood that other aglycons could be employed in the reactions below merely by replacing HO(CH₂)₈COOCH₃ with other alcohols of the formula HOR where R is as defined herein.

Generally, the synthetic processes of this invention start with the disaccharide lactose. This compound is fully hydroxyl protected by reaction with an excess of benzoyl chloride in an inert diluent as shown in reaction (1) below:

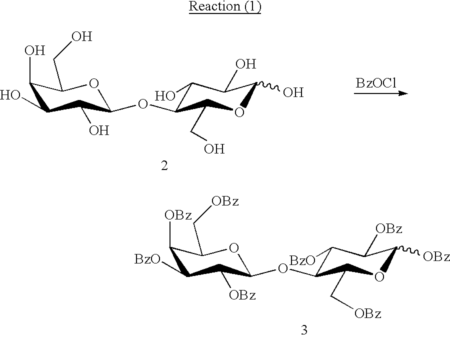

Reaction (1) is preferably conducted by combining lactose, compound 2, with an excess, i.e., at least 8 stoichiometric equivalents, of benzoyl chloride in a suitable inert diluent. Preferably, the amount of benzoyl chloride employed in the reaction is from at least about 8 to about 16 equivalents and more preferably about 12 equivalents.

In one embodiment, an organic base such as triethylamine, diisopropyl-ethylamine, 4-N,N-dimethylaminopyridine and the like is added to the reaction mixture to scavenge the acid generated. In another embodiment, an diluent containing a basic group is employed. In this case, pyridine is employed as the preferred diluent. Preferably, the reaction solution comprises 4-N,N-dimethylaminopyridine in pyridine.

The reaction is conducted under conditions to provide for per-O-benzoyl-β-D-lactoside, compound 3. Preferably, the reaction is conducted at a temperature of from about 20° to about 80° C. and more preferably at about 65° C. for a period of from about 6 to about 48 hours. Specifically, it has been found that maintaining the reaction at about 65° C. significantly reduces reaction time as compared to reaction at room temperature. After reaction completion, methanol is preferably added to the reaction mixture to destroy any unreacted benozyl chloride. The resulting product is recovered by conventional methods including stripping of the solvent, chromatography, crystallization and the like, or can be used directly in the next reaction procedure without purification and/or isolation.

In one preferred embodiment, the reaction mixture is concentrated, dissolved in methylene chloride, washed with water, an acidic aqueous solution and a basic solution until neutral. The resulting product is then dried over sodium sulfate, filtered and is used directly in reaction (2).

In reaction (2), the anomeric benzoyl group is converted to the corresponding α-bromo group as shown below:

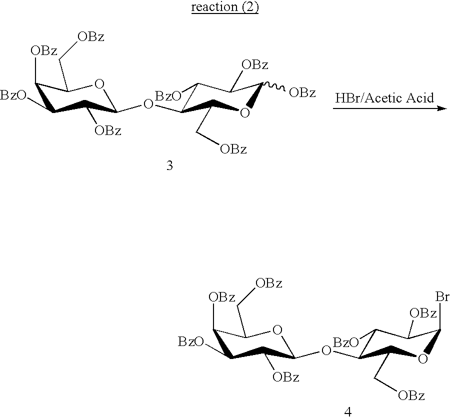

Reaction (2) is preferably conducted by combining per-O-benzoyl-β-D-lactoside, compound 3, with an excess of hydrogen bromide/acetic acid in a suitable diluent. Preferably, the amount of hydrogen bromide employed in the reaction is from at least 1 to about 10 equivalents and more preferably about 4 equivalents.

The reaction is conducted under conditions to provide for 2,3,6,2′,3′,4′,6′-hepta-O-benzoyl-α-D-lactosyl bromide, compound 4. Preferably, the reaction is conducted at a temperature of from about 10° to about 40° C., more preferably from about 10° C. to about 30° C. and still more preferably at about room temperature for a period of from about 6 to about 32 hours. After reaction completion, the resulting product is recovered by conventional methods including stripping of the solvent, chromatography, crystallization and the like, or can be used directly in the next reaction procedure without purification and/or isolation.

In one preferred embodiment, the reaction solution is washed repeatedly with a basic aqueous solution until the pH of the aqueous wash solution is about 7. The organic solution is then concentrated and recovered by conventional methods to provide for compound 4.

2,3,6,2′,3′,4′,6′-Hepta-O-benzoyl-α-D-lactosyl bromide, compound 4, is converted to the 8-methoxycarbonyloctyl-2,3,6,2′,3′,4′,6′-hepta-O-benzoyl-β-D-lactoside, compound 5, as shown in reaction (3) below:

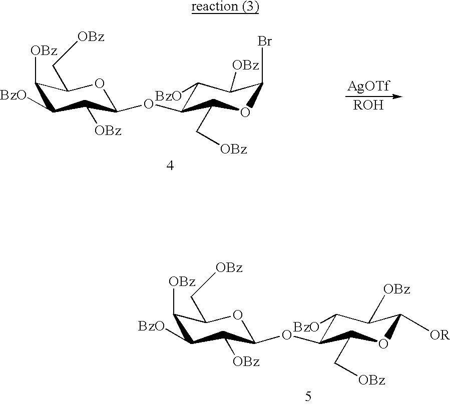

where R is —(CH₂)₈COOCH₃.

Reaction (3) involves the formation of a glycosidic linkage on the anomeric carbon atom of the reducing sugar wherein the benzoyl protected lactosyl bromide, compound 4, is reacted under catalytic conditions well known in the art with an aglycon which possesses one free hydroxyl at the position where the glycosidic linkage is to be established. A large variety of aglycon moieties are known in the art and can be attached with the proper configuration to the anomeric center of the reducing unit.

Reaction (3) is preferably conducted by combining 2,3,6,2′,3′,4′,6′-hepta-O-benzoyl-α-D-lactosyl bromide, compound 4, with an excess of the alcohol ROH (i.e., HO—(CH₂)₈COOCH₃) in a suitable inert diluent. Suitable diluents include, by way of example, chloroform, methylene chloride, and the like. Preferably, the amount of the alcohol employed in the reaction is from at least 1 to about 1.5 equivalents and more preferably about 1.2 equivalents.

The reaction is conducted under conditions to provide for 8-methoxycarbonyloctyl-2,3,6,2′,3′,4′,6′-hepta-O-benzoyl-β-D-lactoside, compound 5. Preferably, the reaction is conducted at a temperature of from about 10° to about 50° C. and more preferably at about room temperature for a period of from about 0.1 to about 10 hours. A stoichiometric excess and preferably about 1.3 equivalents of silver trifluoromethane sulfonate (AgOTf) is employed in the reaction to assist in conversion of the lactosyl bromide to compound 4. In addition, a stoichiometric excess and preferably about 1.1 equivalents of 1,1′,3,3′-tetramethyl urea (TMU) is employed in the reaction to enhance the overall yield of the desired β-D-lactoside. In a particularly preferred embodiment, molecular sieves, e.g., 4 Å molecular sieves, are added to the reaction mixture.

After reaction completion, the reaction solution is preferably neutralized with the addition of a trialkyl amine such as triethylamine, diisopropylethyl amine and the like to provide for compound 5. The resulting product is then recovered by conventional methods including stripping of the solvent, chromatography, crystallization and the like, or can be used directly in the next reaction procedure without purification and/or isolation.

The benzoyl blocking groups on the 8-methoxycarbonyloctyl-2,3,6,2′,3′,4′,6′-hepta-O-benzoyl-β-D-lactoside, compound 5, are then removed by conventional methods as shown in reaction (4) below to provide for the desired 8-methoxycarbonyloctyl-β-D-lactoside, compound 6.

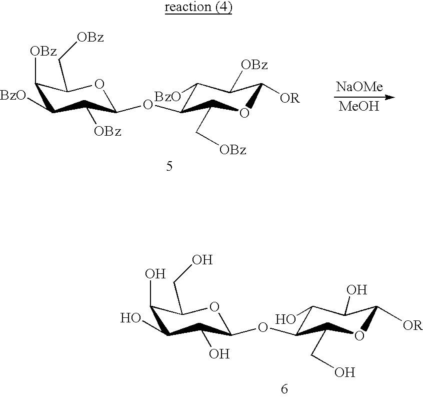

The reaction is conducted under conditions to provide for 8-methoxycarbonyloctyl-β-D-lactoside, compound 6. Preferably, the reaction is conducted using an excess of sodium methoxide in methanol at a temperature of from about 30° to about 60° C. and more preferably at about 45° C. After reaction completion, the reaction solution is neutralized by addition of acetic acid. The resulting product is then recovered by conventional methods including stripping of the solvent, crystallization and the like.

The overall yield from compound 2 to compound 6 can be as high as 75%.

8-Methoxycarbonyloctyl-β-D-lactoside, compound 6, is then converted to 8-methoxycarbonyloctyl-4′,6′-O-benzylidene-β-D-lactoside, compound 7, as shown in reaction (5) below:

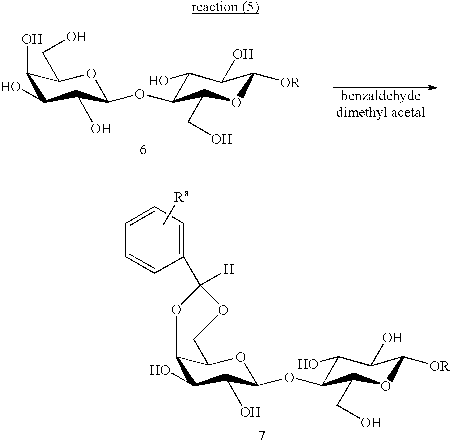

where R and R^{a} are as defined above.

This reaction generally employs a stoichoimetric excess of a benzaldehyde dimethyl acetal wherein the phenyl group of the benzaldehyde moiety is optionally substituted with an alkyl, an alkoxy, an aryl, an alkaryl, an aryloxy, a cyano group or a halo group. The reaction is typically conducted at room temperature in an anhydrous inert solvent, such as acetonitrile, in the presence of an acid catalyst such as p-toluenesulfonic acid to provide an acidic solution preferably with a pH of about 3. The reaction is generally conducted at from about 20° C. to about 60° C. and preferably at about 40° C. and is generally complete in about 12 to 48 hours.

After reaction completion, the reaction solution is preferably neutralized with the addition of a trialkylamine such as triethylamine, diisopropylethyl amine and the like to provide for compound 7. The resulting product is then recovered by conventional methods including stripping of the solvent, chromatography, crystallization and the like, or can be used directly in the next reaction procedure without purification and/or isolation.

The unprotected hydroxyl groups of compound 7 are then benzolyated in the manner of reaction (1) above with the exception that a proportionally smaller amount of the benzoyl halide is required to provide for 2,2′,3,3′,6-penta-O-benzoyl-4′,6′-O-benzylidene-β-D-lactoside, compound 8, as shown in reaction (6) below:

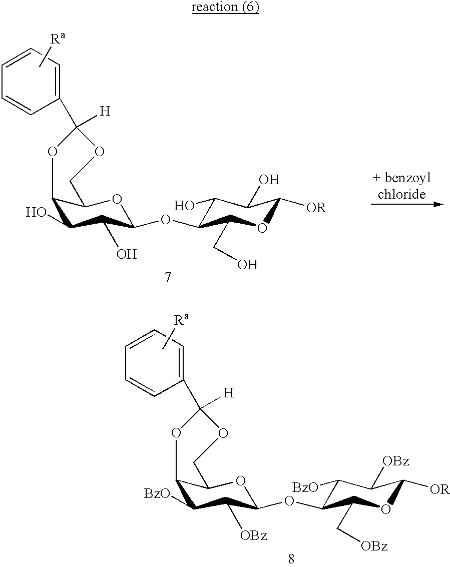

where R and R^{a} are as defined above.

After reaction completion, methanol is preferably added to the reaction mixture to destroy any unreacted benozyl chloride. The resulting product is recovered by conventional methods including stripping of the solvent, chromatography, crystallization and the like, or can be used directly in the next reaction procedure without purification and/or isolation.

The 4,6-O-benzylidene group of compound 8 is then opened regioselectively in the manner described by Liptak, et al.¹² to provide compound 9 having an unblocked hydroxyl group at the 4 position as shown in reaction (7) below:

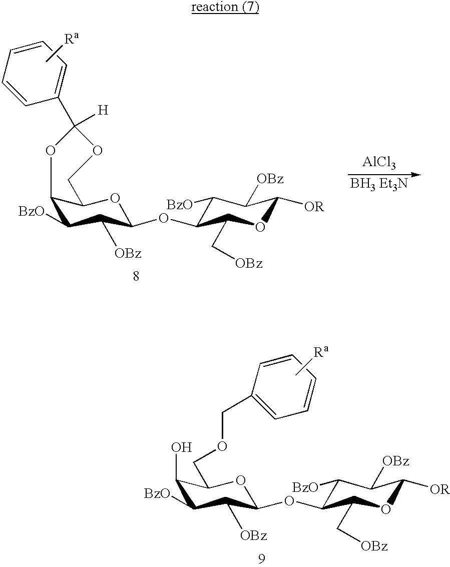

where R and R^{a} are as defined above.

Regioselective opening of the benzylidene group is affected by treatment of compound 8 with an excess of aluminum trichloride/BH₃·Et₃N followed by the dropwise addition of trifluoroacetic acid in a suitable inert diluent such as tetrahydrofuran. The reaction conditions are not critical and the conditions are selected so as to produce compound 9. Preferably, the reaction is conducted at a temperature of from about −20° C. to about 20° C. and more preferably at about 0° C. Upon completion, the reaction mixture is washed with an acidic solution and a basic solution until neutral pH is reached to destroy any unreacted reagents. The resulting product is recovered by conventional methods including stripping of the solvent, chromatography, crystallization and the like, or can be used directly in the next reaction procedure without purification and/or isolation.

The structure of compound 9 can be confirmed by ¹H NMR spectroscopy using trichloroacetyl isocyanate which resulted in the downfield shift of the H-4″ proton signal to 6.65 relative to TMS.

Linkage of the terminal galactose group to compound 9 is accomplished by contacting compound 9 with tetra-O-benzyl-α-chloro galactoside in the presence of TMU and AgOTf under conditions described by Nillson, et al.¹³ which provide for 8-methoxycarbonyloctyl-(2″,3″,4″,6″-tetra-O-benzyl-α-D-galactopyranosyl)(1→4)-(6′-O-benzyl-2′,3′-di-O-benzoyl-β-D-galactopyranosyl)-(1→4)-O-2,3,6-tri-O-benzoyl-β-D-glucopyranoside, compound 9, as shown in reaction (8) below:

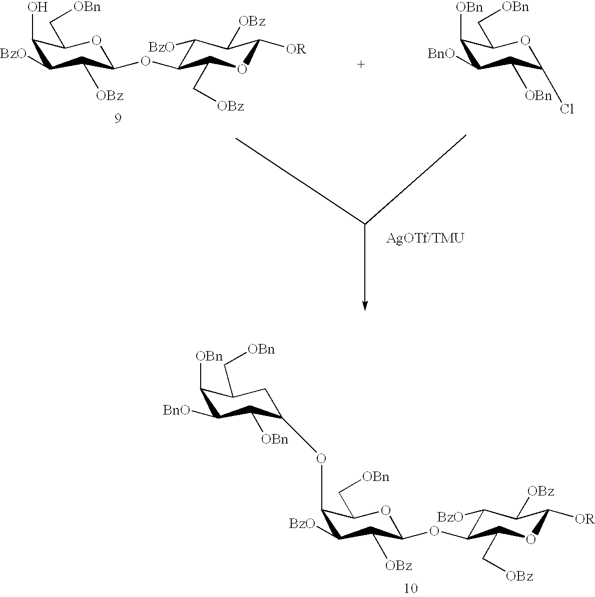

The 4′ hydroxy group of compound 9 and the benzyl protecting groups of the α-chloro tetrabenzyl galactoside direct the reaction to formation of the α(1→4) linkage between the two galactose units.

Preferably, reaction (8) employs from about 1 to about 5 equivalents of the α-chloro tetrabenzylgalactoside per compound 9 and more preferably about 3 equivalents. Additionally, in a preferred embodiment the reaction employs from about 2 to about 4 equivalents of AgOTf per equivalent of disaccharide 9 and about 2 to 4 equivalents of an organic base such as TMU. The reaction mixture is protected from light and the reaction is conducted at a temperature of from about 0° to 60° C. and preferably at room temperature. The reaction is continued until complete as evidenced by thin layer chromatography which is typically from about 16 to 24 hours. Suitable inert diluents include, by way of example, chloroform, methylene chloride, and the like.

The resulting product can then be recovered by conventional methods including stripping of the solvent, chromatography, crystallization and the like, or can be used directly in the next reaction procedure without purification and/or isolation.

After formation of trisaccharide 10, the blocking groups are then removed by conventional methods as shown in FIG. 1 to provide for the desired αGal(1→4)βGal(1→4)Glc-OR compounds including 8-methoxy-carbonyloctyl α-D-galactopyranosyl-(1→4)-O-β-D-galactopyranosyl-(1→4)-O-β-D-glucopyranoside (compound 1). The resulting product can be recovered by conventional methods, including crystallization.

The α-chloro tetrabenzyl galactoside used in reaction (8) can be synthesized directly from commercially available tetrabenzyl thiogalactoside in one pot using NCS and tetraethylammonium chloride (Et₄NCl).

As discussed above, the R aglycon preferably contains a functional group or can be derivatized to contain such a group which can provide for covalent linkage to a solid support. For example, as illustrated by Pinto, et al.¹² and Lemieux, et al.¹¹, the carboxymethyl (—COOCH₃) group of a —(CH₂)₈COOCH₃ aglycon can be converted under conventional conditions to the acyl azide derivative and then covalently linked to a solid support via an amide group. As discussed above, other aglycons which can be used to effect covalent linkage to a solid support can be found in Ekberg, et al.³, Dahmen, et al.⁴, Rana, et al.⁵, Amvam-Zollo, et al.⁶, Paulsen, et al.⁷, Chernnyak, et al.⁸, Fernadez-Santana, et al.⁹ and Lee, et al.¹⁰

Utility

The methods of this invention provide for the synthesis of compounds capable of binding shiga-like toxins (SLTs) and, accordingly, are useful in the treatment of diarrhea mediated by, for example, SLTs expressed by pathogenic E. coli.¹ When so employed, the compound, either by itself or attached to a pharmaceutically acceptable solid support, can be administered as a pharmaceutical composition to a patient suffering from SLT mediated disease conditions. Oral administration of the compound coupled to a pharmaceutically acceptable solid support is preferred whereas, when the compound is not attached to a solid support, administration rectally is the preferred route.

These compounds can also be used in diagnostic assays for determining the presence of SLTs in a biological sample. For example, these compounds, appropriately labeled, can be used in a competitive assay to determine the amount and/or presence of SLTs in a compound. Suitable detectable labels include, by way of example, radiolabels, fluorescent labels, magnetic labels, enzymes, and the like. Attachment of a detectable label to these compounds is achieved via conventional methods well known in the art.

Alternatively, the trisaccharide, containing an appropriate aglycon, can be attached to a solid support in the manner described above to provide a means to remove SLTs from a sample. After contact, the solid support is freed from the biological sample by conventional means such as washing, centrifugation, etc. Additionally, such solid supports can be used in a conventional ELISA assay to detect for the presence of SLTs in a biological sample such as a stool sample.

### EXAMPLES

The following examples are set forth to illustrate the claimed invention and are not to be construed as a limitation thereof.

Unless otherwise stated, all temperatures are in degrees Celcius. Also, in these examples as well as in FIG. 1, unless otherwise defined below, the abbreviations employed have their generally accepted meanings:

| | | |
|---|---|---|
| Å | = | Angstroms |
| AgOTf | = | silver trifluoromethane sulfonate |
| Bn | = | benzyl |
| Bz | = | benzoyl |
| C₇H₈ | = | toluene |
| cm | = | centimeter |
| d | = | doublet |
| DCM | = | dichloromethane |
| DMAP | = | dimethylaminopyridine |
| EDTA | = | ethylene diamine tetraacetic acid |
| EtOAc | = | ethyl acetate |
| g | = | gram |
| h | = | hour |
| kg | = | kilogram |
| L | = | liter |
| m | = | multiplet |
| MeCN | = | acetonitrile |
| MeOH | = | methanol |
| N | = | normal |
| p-TsOH | = | para-toluene sulfonic acid |
| s | = | singlet |
| t | = | triplet |
| TEA | = | triethylamine. |
| TFA | = | trifluoroacetic acid |
| THF | = | tetrahydrofuran |
| TLC | = | thin layer chromatography |
| TMU | = | tetramethyl urea |
| μ | = | micron |
| v/v | = | volume to volume |
| w/w | = | weight to weight |

All solvents were commercial grade and used without purification or drying. Melting points were measured with Fisher John instrument and are uncorrected. NMR spectra were recorded on 300 MHz (Bruker AMX-300 MHz) with either internal (CH₃)₄Si (δ=7.26, CDCl₃) or DOH (δ=4.80, D₂O) and ¹³C NMR at 75 MHz. FTIR spectra were recorded on a PE, Spectrum-1000 instrument. High-resolution mass spectroscopy and optical rotation data was obtained from the spectral services of the Department of Chemistry, University of Alberta, Edmonton, Canada.

The synthesis scheme employed in the following examples is illustrated in FIG. 1.

### Example 1

### Synthesis of 1,2,3,6,2′,3′,4′,6′-O-Octabenzoyl lactoside (Compound 3)

Commercially available lactose (400 g, 1.10 mol) was stirred in pyridine (4.5 kg) and DMAP (135 g, 1.10 mol) at ambient temperature. Benzoyl chloride (1.7 kg, 12.3 mol) was added to the reaction vessel and the reaction was stirred at 65° C. The progress of the reaction was monitored by TLC (C₇H₈:EtOAc; 85:15, product α-anomer R_{f}=0.58, β-anomer R_{f}=0.51). Excess benzoyl chloride was quenched by the addition of MeOH (250 g) and the reaction mixture concentrated to a syrup. The crude syrup was re-dissolved in DCM and washed with water, 5% HCl, 6% NaHCO₃ and finally with water. The organic fraction was dried over sodium sulfate and the stock solution was carried forward to the next step.

### Example 2

### Synthesis of 2,3,6,2′,3′,4′,6′-O-Heptabenzoyl-α-D-lactosyl bromide (Compound 4)

To the stock solution of 1,2,3,6,2′,3′,4′,6′-O-octabenzoyl lactoside (compound 3) was added 30% w/w HBr/acetic acid (1110 g, 4.12 mol) and the reaction was stirred at ambient temperature. The progress of the reaction was determined by TLC (C₇H₈:EtOAc; 85:15, product R_{f}=0.53). The organic layer was washed with water, 6% NaHCO₃, and 6% brine solution. The organic layer was evaporated under reduced pressure to give the desired product (compound 4) which was taken directly to the next step.

### Example 3

### Synthesis of 8-Methoxycarbonyloctyl-β-D-lactoside (Compound 6)

Compound 4 (1300 g, 1.17 mol), dissolved in DCM (2 kg), was added to a reaction vessel containing DCM (10 kg), 4 Å molecular sieves (1.5 kg), 8-methoxycarbonyloctanol (263 g, 1.40 mol), TMU (147 g, 1.26 mol) and AgOTf (389 g, 1.51 mol). The reaction mixture was stirred at ambient temperature until TLC (C₇H₈:EtOAc, 85:15 v/v, product R_{f}=0.48) showed complete consumption of the starting material. The reaction mixture was neutralized with TEA to pH 6-7. The mixture was filtered over Celite and then rinsed with DCM (3 kg). The filtrate was washed with water (×2) and the organic layer was evaporated under reduced pressure to give a syrup. The crude syrup was extracted with hexanes for 30 minutes, decanted and the residual hexanes evaporated to furnish a solid residue which was dried under high vacuum.

The residue was dissolved in methanol (5.5 kg) followed by addition of 1N anhydrous sodium methoxide (300 g) to pH>11. The reaction was performed at 45° C. until TLC (CHCl₃:MeOH:H₂O, 65:35:8, product R_{f}=0.48) showed complete consumption of the starting material. The reaction mixture was neutralized by addition of glacial acetic acid to pH 5-7. The solvent was removed by evaporation under reduced pressure. The crude product was extracted with hexanes, decanted and evaporated under reduced pressure to give a solid. The compound was precipitated using MeOH/EtOAc at 45° C. for 3 hours followed by cooling overnight. The crystals were filtered, washed with EtOAc, and dried under high vacuum to furnish the desired product (compound 6) (75% yield, 400 g). [α]_{D}−1.41° (c 0.5, H₂O); mp 158-160° C.; ¹H NMR (D₂O/CD₃OD) δ4.51 (d, J=8 Hz, 1H, H-1′), 4.47 (d, J=8 Hz, 1H, H-1), 3.71 (s, 3H, —OCH₃), 2.40 (t, 2H, —CH₂CO), 1.20-1.78 (m, 12H, —CH₂—); ¹³C NMR (D₂O/CD₃OD) δ177.7, 103.8, 103.0, 79.3, 76.1, 75.5, 75.3, 73.7, 73.4, 71.7, 71.2, 69.4, 61.7, 60.9, 52.5, 34.4, 29.6, 29.2, 29.1, 29.0, 25.8, 25.1; IR (KBr) 3395, 2920, 2851, 1729, 1438, 1086, 1064, 1037 cm⁻; HRMS calcd for.C₂₂H₄₀O₁₃ [M+H]⁺ 513.2547, found 513.2536.

### Example 4

### Synthesis of 8-Methoxycarbonyloctyl 4′,6′-O-benzylidine-β-D-lactoside (Compound 7)

8-Methoxycarbonyloctyl-β-D-lactoside (compound 6) (340 g, 0.66 mol) along with benzaldehyde dimethyl acetal (322 g, 2.10 mol) was stirred in MeCN (5.7 kg). The pH of the suspension was adjusted to 3 by addition of p-TsOH (14 g, 60 mmol). The reaction was heated at 40° C. for 16-24 h until completion as indicated by TLC (CHCl₃:MeOH:H₂O; 65:35:8, product R_{f}=0.77). The reaction was neutralized by addition of TEA, filtered and the solvents evaporated under vacuum to furnish the desired product which was directly taken to the next step without any further purification.

### Example 5

### Synthesis of 8-Methoxycarbonyloctyl 2,3,6,2′,3′-penta-O-benzoyl-4′,6′-O-benzylidene-β-D-lactoside (Compound 8)

8-Methoxycarbonyloctyl 4′,6′-O-benzylidine-β-D-lactoside (compound 7) was stirred in pyridine (4.7 kg), and DMAP (84 g, 0.68 mol) at room temperature. Benzoyl chloride (742 g, 5.28 mol) was added dropwise and the reaction was stirred overnight. The progress of the reaction was monitored by TLC (C₇H₈:EtOAc; 85:15, product R_{f}=0.35). Excess benzoyl chloride was slowly quenched by addition of MeOH (250 g). The solvent was removed under reduced pressure and the residue was re-dissolved in DCM. The reaction mixture was washed with water, 5% HCl, 6% NaHCO₃ and water until neutral pH. The solvent was removed by evaporation to give the desired product (compound 8), which was crystallized from MeOH (85% yield, 632 g). [α]_{D}+98.18° (c 1.7, CHCl₃); mp 164-166° C.; ¹H NMR (CDCl₃) δ7.10-8.07 (m, 30H, C₆H₅), 5.29 (s, 1H, benzylidene), 4.85 (d, J=8 Hz, 1H, H-1′), 4.66 (d, J=8 Hz, 1H, H-1), 3.65 (s, 3H, O═C—O—CH₃), 2.21 (m, 2H, —CH₂CO), 0.92-1.7 (m, 12H, —CH₂—); ¹³C NMR (CDCI₃) δ174.2, 166.0, 165.6, 165.3, 165.0, 164.8, 137.4, 133.1, 133.0, 132.9, 129.9, 129.8, 129.7, 129.65, 129.6, 129.5, 128.9, 128.8, 128.7, 128.4, 128.3, 128.28, 128.26, 128.2, 127.9, 126.3, 101.4, 100.7, 100.6, 76.8, 76.5, 74.0, 73.0, 72.7, 72.6, 72.3, 69.4, 66.4, 51.4, 50.8, 34.0, 29.2, 29.0, 28.9, 25.6, 24.8; IR (KBr) 3433, 3064, 2936, 1724, 1602, 1452, 1274, 1070, 708 cm⁻¹; HRMS calcd for.C₆₄H₆₄O₁₈ [M+Na]⁺ 1143.3990, found 1143.4004.

### Example 6

### Synthesis of 8-Methoxycarbonyloctyl 2,3,6,2′,3′-penta-O-benzoyl-6′-O-benzyl-β-D-lactoside (Compound 9)

Aluminum chloride (204 g, 1.5 mol) was slowly added to THF (3.3 kg) at 0° C. After the reaction had subsided, BH₃.Et₃N (112 g, 1.5 mol) and compound 8 (340 g, 0.3 mol) were added. Once the reaction stabilized at 0° C., TFA (175 g, 1.5 mol) was added dropwise. The reaction was allowed to proceed at 0° C. and monitored by TLC (C₇H₈:EtOAc:MeOH; 85:15:2, product R_{f}=0.63). Upon completion of the reaction as detected by TLC, the mixture was extracted twice with 5% H₂SO₄, and 6% NaHCO₃ solution until the pH was neutral. The solvents were removed by evaporation under vacuum to give a solid residue, which was crystallized from MeOH, to give the desired product (compound 9) (86% yield, 292 g). [α]_{D}+55.30° (c 1.6, CHCl₃); mp 80-82° C.; ¹H NMR (CDCl₃) δ7.17-8.03 (m, 30H, C₆H₅), 4.76 (d, J=8 Hz, 1H, H-1′), 4.65 (d, J=8 Hz, 1H, H-1), 3.64 (s, 3H, O═C—O—CH₃), 2.21 (m, 2H, —CH₂CO), 0.92-1.54 (m, 12H, —CH₂—); ¹³C NMR (CDCl₃) δ174.2, 165.7, 165.6, 165.3, 165.2, 165.0, 137.6, 133.2, 133.1, 129.9, 129.8, 129.77, 129.7, 129.67, 129.65, 129.4, 129.0, 128.8, 128.4, 128.38, 128.3, 128.29, 127.7, 127.4, 101.2, 100.8, 76.3, 73.5, 73.2, 72.9, 71.9, 70.0, 69.9, 67.3, 51.4, 34.0, 29.2, 28.95, 28.9, 25.6, 24.8; IR (KBr) 3448, 3066, 2930, 1729, 1602, 1452, 1274, 1107, 708 cm⁻¹; HRMS calcd for C₆₄H₆₆O₁₈ [M+Na]⁺ 1145.4146, found 1145.4152.

### Example 7

### Synthesis of Blocked P^{k}-trisaccharide methyl ester (Compound 10)

To a solution of compound 9 (312 g, 0.28 mol) in DCM (5.9 kg) protected from light and moisture was added 4 Å molecular sieves (312 g), TMU (84 g, 0.72 mol) and AgOTf (173 g, 0.67 mol). The mixture was stirred for 1 hour and α-chloro tetrabenzylgalactose (470 g, 0.84 mol) was charged to the reaction flask. The reaction was allowed to proceed at room temperature for 16-24 h and the progress of the reaction was monitored by TLC (C₇H₈:EtOAc; 85:15, product R_{f}=0.5). The reaction mixture was filtered and the filtrate was extracted with EDTA, and water. The organic layer was dried, filtered and the solvents evaporated under vacuum. The crude product (compound 10) was taken directly to the next step without any further purification.

### Example 8

### Synthesis of 6′,2″,3″,4″,6″ Penta-O-benzyl P^{k}-trisaccharide methyl ester (Compound 11)

To a solution of the crude ester (compound 10) (234 g) in MeOH (3.0 kg) was added sufficient amount of 1N anhydrous sodium methoxide (164 g) to pH˜12. The reaction mixture was stirred at 45° C. and the progress of the reaction was monitored by TLC (DCM:MeOH; 95:5, product R_{f}=0.25). The reaction mixture was neutralized by the addition of glacial acetic acid (pH˜5-7). The solvent was evaporated and the residue was extracted with EtOAc, washed with water, and 6% brine solution. The organic layer was evaporated and the crude product was purified by column chromatography. The fractions containing the purified product were pooled together and the solvents evaporated under vacuum to furnish the product (compound 11) in 75% yield (234 g) as a syrup. [α]_{D}+8.75° (c 2.7, CHCl₃); ¹H NMR (CDCl₃) δ7.10-8.15 (m, 25H, C₆H₅), 3.65 (s, 3H, O═C—O—CH₃), 2.29 (t, J=8 Hz, 2H, —CH₂CO), 1.20-1.70 (m, 12H, —CH₂—); ¹³C NMR (CDCl₃) δ174.2, 138.3, 138.0, 137.7, 137.3, 128.6, 128.5, 128.4, 128.38, 128.34, 128.3, 128.2, 127.9, 127.87, 127.84, 127.8, 127.7, 127.5, 104.2, 102.5, 100.5, 84.0, 80.9, 78.7, 76.5, 76.3, 74.9, 74.5, 74.4, 74.2, 74.1, 73.9, 73.8, 73.5, 73.3, 73.1, 71.7, 71.3, 70.1, 69.2, 63.2, 51.4, 34.0, 29.5, 29.1, 29.08, 29.0, 25.8, 24.8; IR (KBr) 3448, 3054, 2930, 2305, 1732, 1454, 1265, 1097, 896, 739 cm⁻¹; HRMS calcd for C₆₃H₈₀O₁₈ [M+Na]⁺ 1147.5242, found 1147.5241.

### Example 9

### Synthesis of P^{k}-trisaccharide methyl ester (Compound 1)

To a nitrogen purged solution of compound 11 (120 g, 0.1 mol) in MeOH (4.9 kg) was added 10% Pd/C (70 g). The mixture was stirred and H₂ gas was bubbled through the solution for 5-8 h. The progress of the reaction was monitored by TLC (CHCl₃:MeOH:H₂O; 65:35:8, product R_{f}=0.67). Upon completion of the reaction, the catalyst was removed by filtration and the solvent was removed by evaporation under vacuum. The solid residue was crystallized from MeOH/n-propanol mixture to give compound 1 in 80% yield (57 g). [α]_{D}+46.98° (c 1.9, H₂O); mp 156-158° C.; ¹H NMR (D₂O) δ4.95 (d, J_{1″,2″}=4.0 Hz, 1H, H-1″), 4.51 (d, J_{1′,2′}=7.8 Hz, 1H, H-1′), 4.48 (d, J_{1,2}=8.1 Hz, 1H, H-1), 4.35 (ψt, 1H, H-5″), 4.04 (d, 1H, H-4′), 4.03 (d, 1H, H-4″), 3.99 (1H, H-6a), 3.93 (1H, H-6a′), 3.91 (1H, H-3″), 3.84 (J=10.5 Hz, 2H, H-2″, H-6b′), 3.83 (1H, H-6b), 3.79 (1H, H-5′), 3.74 (2H, H-6a″, H-3), 3.70 (m, 1H, H-6b″), 3.69 (s, 3H, —OCH₃), 3.65 (1H, H-4), 3.64 (dd, J=9.8 Hz, H-3), 3.58 (2H, H-5, H-2′), 3.30 (dd, J=9.2 Hz, 1H, H-2), 2.39 (m, 2H, —CH₂CO), 1.30-1.80 (m, 12H, —CH₂CO), 1.30-1.80 (m, 12H, —(CH₂)—); ¹³C NMR (D₂O) δ178.1, 103.5 (1C, C-1′), 102.3 (1C, C-1′), 102.3 (1C, C-1), 100.6 (1C, C-1″), 79.0, 77.7, 75.7, 75.1, 74.8, 73.2, 72.5, 71.2, 71.1, 71.0, 69.4, 69.2, 68.9, 60.8, 60.7, 60.4, 52.4, 34.0, 29.0, 28.7, 28.6, 28.4, 25.3, 24.6; IR (KBr) 3401, 2930, 1736, 1438, 1073, 810, 700, 545 cm¹; HRMS calcd for C₂₈H₅₀O₁₈ [M+Na]⁺ 697.2894, found 697.2903.

Although the foregoing invention has been described in some detail by way of example, it will be apparent that certain modifications may be practiced within the scope of the appended claims.

What is claimed is:

1. A process for preparing αGal(1→4)βGal(1→4)Glc-OR compounds which process comprises:
(a) contacting β-R-lactoside represented by the formula: 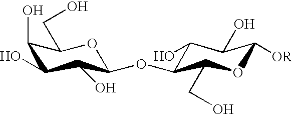
with at least a stoichiometric amount of a benzaldehyde dimethylacetal under conditions to provide for β-R-4′,6′-O-benzylidine lactoside of the formula: 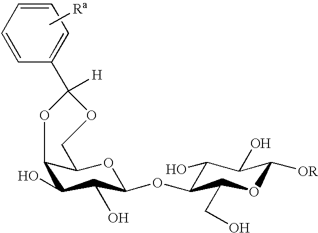
where R is an aglycon of at least 1 carbon atoms and R^{a} is selected from the group consisting of hydrogen, alkyl, alkoxy, aryl, aryloxy, cyano and halo;
(b) acylating the compound produced in (a) above with at least 5 equivalents of an acyl halide under conditions to provide for β-R-4′,6′-O-benzylidine-2,2′,3,6,6′-pentaacyloxy-lactoside of the formula: 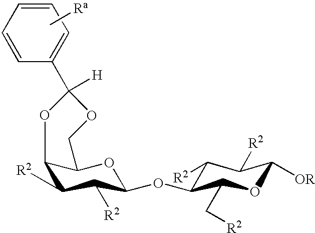
wherein R and R^{a} are as defined above and each R² is an acyloxy group;
(c) opening the benzylidine group from the compound produced in (b) above to provide for the β-R-2,2′,3,3′,6-pentaacyloxy-6′-O-benzyl lactoside of the formula: 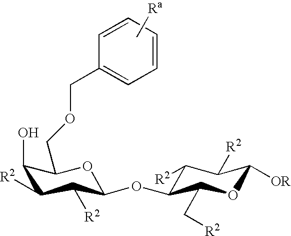
where R, R^{a} and R² are as defined above;
(d) contacting the compound produced in (c) above with α-chloro 2,3,4,6-tetra-O-benzyl-D-galactose under conditions to provide for a compound of the formula: 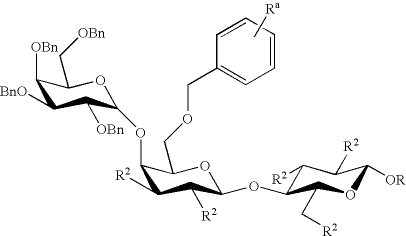
where R, R^{a} and R² are as defined above;
(e) removing each of the benzyl and R² protecting groups in the compound produced in (f) above under conditions to provide for αGal(1→4)βGal(1→4)Glc-OR which is represented by the formula: 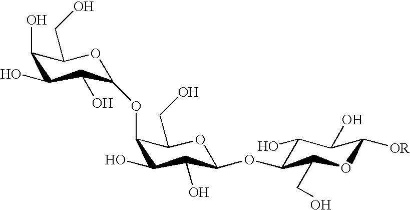
where R is as defined above.

2. The process of claim 1 wherein β-R-lactoside, represented by the formula:
where R is as defined above, is prepared by the following process: 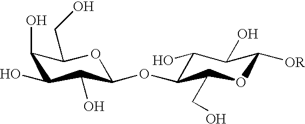
(i) contacting lactose of the formula: 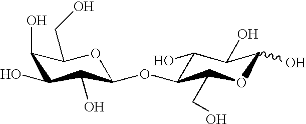
with at least 8 equivalents of benzoyl halide under conditions to provide for β-per-O-benzoyl-lactoside of the formula: 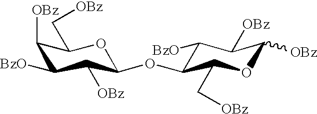
(ii) contacting the β-per-O-benzoyl-lactoside prepared in (i) above with at least a stoichiometric amount of hydrogen bromide under conditions to form the 1-α-bromo derivative of the formula: 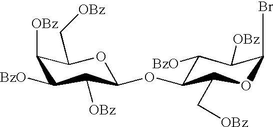
(iii) contacting the compound produced in (ii) above with a compound of the formula ROH under conditions to provide for β-R-2,2′,3,3′,4′,6,6′-hepta-O-benzoyl lactoside of the formula; 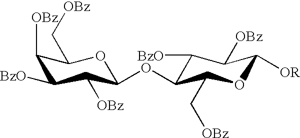
wherein R is an aglycon of at least 1 carbon atom; and
(iv) contacting the compound produced in (iii) above under conditions to debenzoylate said compound to provide for β-R-lactoside.

3. The process of either claim 1 or 2 wherein R contains from 1 to 20 carbon atoms.

4. The process of either claim 1 or 2 wherein each R² is —O-benzoyl.

5. A process for the synthesis of αGal(1→4)βGal(1→4)Glc-O(CH₂)₈—COOCH₃ which process comprises:
(a) contacting lactose of the formula: 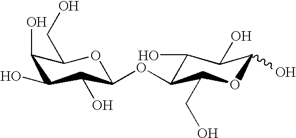
with at least 8 equivalents of benzoyl halide under conditions to provide for β-per-O-benzoyl-lactoside of the formula: 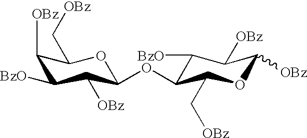
(b) contacting the β-per-O-benzoyl-lactoside prepared in (a) above with at least a stoichiometric amount of hydrogen bromide under conditions to form the 1-α-bromo derivative of the formula: 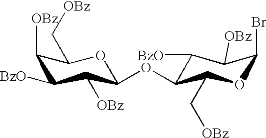
(c) contacting the compound produced in (b) above with at least a stoichiometeric amount of a compound of the formula ROH under conditions to provide for β-OR-2,2′,3,3′,4′,6,6′-hepta-O-benzoyl lactoside of the formula: 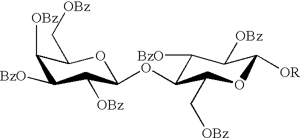
wherein R is —(CH₂)₈COOCH₃;
(d) contacting the compound produced in (c) above under conditions to debenzoylate said compound to provide for β-R-lactoside of the formula: 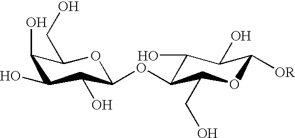
wherein R is as defined above;
(e) contacting β-R-lactoside produced in (d) with at least a stoichiometric amount of a benzaldehyde dimethylacetal under conditions to provide for β-R-4′,6′-O-benzylidine lactoside of the formula: 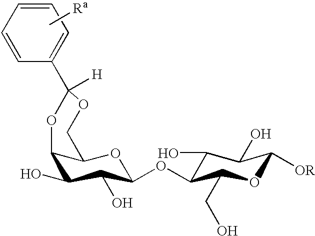
where R is an aglycon of at least 1 carbon atoms and R^{a} is selected from the group consisting of hydrogen, alkyl, alkoxy, aryl, aryloxy, cyano and halo;
(f) acylating the compound produced in (e) above with at least 5 equivalents of an acyl halide under conditions to provide for β-R-4′,6′-O-benzylidine-2,2′,3,6,6′-pentaacyloxy-lactoside of the formula: 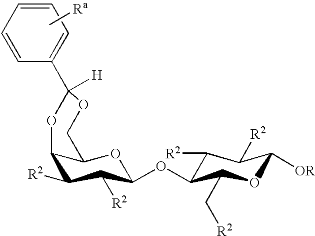
wherein R and R^{a} are as defined above and each R² is an acyloxy group;
(g) opening the benzylidine group from the compound produced in (f) above to provide for the β-R-2,2′,3,3′,6-pentaacyloxy-6′-O-benzyl lactoside of the formula: 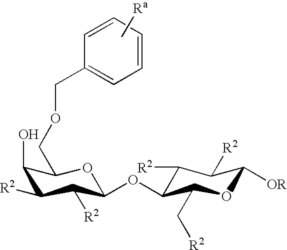
where R, R^{a} and R² are as defined above;
(h) contacting the compound produced in (g) above with α-chloro 2,3,4,6-tetra-O-benzyl-D-galactose under conditions to provide for a compound of the formula: 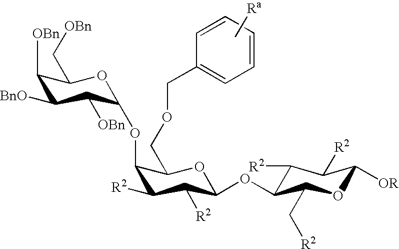
where R, R^{a} and R² are as defined above;
(i) removing each of the benzyl and R² protecting groups in the compound produced in (h) above under conditions to provide for αGal(1→4)βGal(1→4)Glc-OR which is represented by the formula: 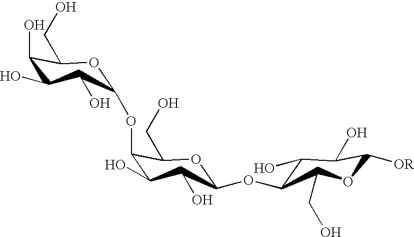
where R is as defined above.
